(19) United States
(12) Patent Application Publication
(10) Pub. No.: US 2013/0122029 A1
(43) Pub. Date: May 16, 2013

(54) CANCER VACCINE

(71) Applicants: Chie Kudo, Tokyo (JP); Yutaka Kawakami, Tokyo (JP)

(72) Inventors: Chie Kudo, Tokyo (JP); Yutaka Kawakami, Tokyo (JP)

(73) Assignee: KEIO UNIVERSITY, Minato-ku (JP)

(21) Appl. No.: 13/637,411

(22) PCT Filed: Mar. 29, 2011
(86) PCT No.: PCT/JP2011/057777

§ 371 Date: Jan. 23, 2013

(30) Foreign Application Priority Data
Mar. 30, 2010 (JP) 2010-078625

(51) Int. Cl. C07K 7/06; A61K 35/14; A61K 39/00

(52) U.S. Cl. CPC C07K 7/06; A61K 39/0011; A61K 35/17
USPC 424/185.1; 530/328; 435/325

(57) ABSTRACT

The present invention is directed to provide a cancer vaccine used for preventing and treating cancers. Provided are a peptide consisting of a sequence of SEQ ID No. 1 (KMHIRSHTL) or SEQ ID No. 2 (RTFSRMSLL) and capable of efficiently inducing cancer immunity, an antigen-presenting cell presenting this peptide on its cell surface, a T cell induced by this antigen-presenting cell as well as a cancer vaccine containing these peptides, an expression vector capable of expressing either of these peptides, the antigen-presenting cell presenting either of these peptides or the T cell induced by the antigen-presenting cell, and a method of treating and preventing cancers using the cancer vaccine.

### CROSS REFERENCE TO RELATED APPLICATION

This application claims the benefit of Japanese Patent Application No. 2010-78625 filed on Mar. 30, 2010, the entire disclosure of which is hereby incorporated by reference.

### TECHNICAL FIELD

The present invention relates to cancer vaccines for prophylactic and therapeutic treatment of cancers.

### BACKGROUND ART

Recent advances in the cancer immunology have considerably elucidated how immune cells recognize cancer antigens. According to this, dendritic cells (DCs) as antigen-presenting cells present, on their surface, antigenic peptides consisting of 8 to 10 amino acids, which have been produced during intracellular degradation of proteins that are expressed in cancer cells, in conjunction with major histocompatibility complex (MHC; in humans MHC is known as the human leukocyte antigen or HLA) molecules. These antigenic peptides bound to the HLA class I molecules on the dendritic cells are recognized by cytotoxic T lymphocytes (CTLs). The CTLs are activated and proliferate to become capable of killing the cancer cells having the protein from which the antigenic peptide was derived when they infiltrate the cancer (e.g., Arch. Surg. (1990) 125: 200-205).

Vaccines have been developed for therapies against cancer based on this mechanism. For example, dendritic cells that present an antigenic peptide derived from a cancer-specific protein on its surface may be produced and expanded in vitro, which may be administered to a cancer patient. Alternatively, the cytotoxic T lymphocytes educated by the dendritic cells may be administered and the cancer immunity is thus induced in the body of a cancer patient. When the cancer-specific protein is administered to the cancer patient, all processes of the mechanism of the cancer immunity are induced in the body of the patient (see, e.g., Science (1991) 254: 1643-1647, J. Exp. Med. (1996) 183: 1185-1192, J. Immunol. (1999) 163: 4994-5004, Proc. Natl. Acad. Sci. USA (1995) 92: 432-436, Science (1995) 269: 1281-1284, and J. Exp. Med. (1997) 186: 785-793).

Only few cancer-specific proteins are, however, known to effectively induce the cancer immunity against certain cancers.

Accordingly, the present invention was directed to provide peptides capable of effectively inducing the cancer immunity, compositions containing the peptide, antigen-presenting cells that present the peptide, T cells stimulated by the antigen-presenting cell, cancer vaccines using the peptide or the cell, and methods of treating a cancer patient using them.

### DISCLOSURE OF THE INVENTION

A peptide according to the present invention consists of the sequence of KMHIRSHTL (SEQ ID No. 1) or RTFSRMSLL (SEQ ID No. 2). The scope of the present invention also includes antigen-presenting cells presenting one of these peptides, and T cells that are induced by the antigen-presenting cells and capable of recognizing a cancer cell expressing a Snail antigen. The T cells are preferably cytotoxic T lymphocytes. The cancer cell expressing the Snail antigen is preferably a pancreatic cancer cell, a melanoma cell, a leukemia cell, or a colon cancer cell.

A cancer vaccine according to the present invention comprises at least one of either or both of the peptides of the SEQ ID Nos. 1 and 2, expression vectors capable of expressing the peptide having the SEQ ID Nos. 1 and 2, respectively, antigen-presenting cells presenting the peptides consisting of the sequences of the SEQ ID Nos. 1 and 2, respectively, on their cell surface, and the aforementioned T cells.

The cancer vaccine comprising either or both of the peptides of the SEQ ID Nos. 1 and 2 according to the present invention may also comprise a cancer antigen peptide other than these peptides.

In addition, the cancer vaccine according to the present invention is preferably against the cancer cells expressing a Snail protein.

A method of treating and preventing a cancer according to the present invention comprises applying the cancer vaccine according to the present invention to human and vertebrate animals other than human.

The term “cancer” as used herein refers to neoplasms of any origin, including carcinomas of epithelial origin, tumors of non epithelial origin, and blood cancers.

A gene with the accession No. NM_{—}005985 (SEQ ID No. 3) is herein referred to as a human snail gene. The term “snail gene” without any limitation of the animal species includes human genes as well as their homologs and orthologs in other animal species. A protein with the accession No. NP_{—}005976 (SEQ ID No. 4) is herein referred to as a human Snail protein. The term “Snail protein” without any limitation of the animal species include human proteins as well as their homologs and orthologs in other animal species.

### BRIEF DESCRIPTION OF THE DRAWINGS

FIG. 1 is a figure showing (A) expression of snail gene in normal tissues of healthy persons and (B) expression of snail gene in cancer tissues of cancer patients and normal tissues of the same patient, in one example of the present invention.

FIG. 2 is a figure showing expression of snail gene in human pancreatic cancer cell lines, human melanoma cell lines, human leukemia cell lines, and human colon cancer cell lines, in one example of the present invention.

FIG. 3 shows graphs of the amount of gamma interferon produced by co-culturing CTLs obtained by culturing HLA-A24-positive peripheral blood monocytes from healthy persons with stimulation with Snail 1, 2, 3 and HERV-H env and NY-ESO-1 peptides, with HLA-A24-positive antigen-presenting cells in the presence of 0.1, 1 or 10 μg/ml of the peptides, in one example of the present invention.

FIG. 4 shows graphs of measurements of tumor-specific cytotoxicity elicited when CTLs obtained by culturing HLA-A24-positive peripheral blood monocytes from healthy persons with stimulation with Snail 3 or NY-ESO-1 were contacted with a tumor cell expressing snail, in one example of the present invention.

FIG. 5 shows graphs of the amount of gamma interferon produced by co-culturing CTLs obtained by culturing HLA-A02-positive peripheral blood monocytes from healthy persons with stimulation with Snail 1, 2, 3 and HERV-H env and NY-ESO-1 peptides, with HLA-A02-positive antigen-presenting cells in the presence of 10 μg/ml of the peptides, in one example of the present invention.

### DETAILED DESCRIPTION OF EMBODIMENTS

Embodiments of the present invention that were completed based on the aforementioned findings are described below in detail in reference to Examples. The present invention is, however, not limited to these Examples.

Unless otherwise noted in embodiments and examples, all procedures used are as described in standard protocols such as J. Sambrook, E. F. Fritsch & T. Maniatis (Ed.), Molecular cloning, a laboratory manual (3rd edition), Cold Spring Harbor Press, Cold Spring Harbor, New York (2001); F. M. Ausubel, R. Brent, R. E. Kingston, D. D. Moore, J. G. Seidman, J. A. Smith, K. Struhl (Ed.), Current Protocols in Molecular Biology, John Wiley & Sons Ltd., with or without modifications or changes. In addition, unless otherwise noted, a commercial reagent kit or a measurement instrument, if any, is used as described according to protocols attached thereto.

The above and further objects, features, advantages, and ideas of the present invention are apparent to those skilled in the art from consideration of the detailed description of this specification. Furthermore, those skilled in the art can easily reproduce the present invention from these descriptions. The mode(s) and specific example(s) described below represent a preferable embodiment of the present invention, which is given for the purpose of illustration or description. The present invention is not limited thereto. It is obvious to those skilled in the art that various modifications may be made according to the descriptions of the present specification without departing from the spirit and scope of the present invention disclosed herein.

### ==Cancer Vaccines==

When a peptide of the SEQ ID No. 1 or 2, which is a part of the amino acid sequence of a Snail protein, is added to dendritic cells as antigen-presenting cells, the peptide binds to an HLA class I molecule and is presented on the cell surface. The complex is then recognized by cytotoxic T lymphocytes. This results in the induction of the cytotoxic T lymphocytes specific to the Snail protein. The cytotoxic T lymphocytes established by the stimulation with these peptides can efficiently recognize the cancer cells expressing the Snail protein. Accordingly, the following can be used as cancer vaccines for prophylactic and therapeutic treatment of cancers: the peptide having the SEQ ID Nos. 1 and/or the peptide having the SEQ ID Nos. 2, antigen-presenting cells presenting the peptide(s) on their cell surface, and cytotoxic T lymphocytes that are induced by the antigen-presenting cells and recognize cancer cells expressing a Snail antigen.

### ==Administration of Cancer Vaccine==

Methods for administering, as a cancer vaccine, cancer-specific antigens, cancer antigen-presenting cells or cytotoxic T lymphocytes reactive to a cancer antigen, to a cancer patient have been developed. The cancer to be treated or prevented with a cancer vaccine using a partial peptide of the Snail protein is not specifically limited as long as it expresses the Snail protein, and may be any of solid and blood cancers, including neuroma, kidney cancer, liver cancer, pancreatic cancer, sarcoma, colon cancer, melanoma, lung cancer, esophageal cancer, uterine cancer, testicular cancer, ovarian cancer, leukemia, lymphoma and myeloma. The cancer is preferably pancreatic cancer, melanoma, leukemia, or colon cancer.

The subject of which cancer is to be treated or prevented with the cancer vaccine according to the present invention is not specifically limited as long as it is a vertebrate animal suffering from the aforementioned cancer. The subject may be a human or a non-human animal.

The cancer vaccines described below may be administered alone or in combination, or alternatively, one or more of them may be administered in combination with a cancer vaccine which is not described herein.

[Cancer Vaccine containing Peptide(s)]

The cancer vaccine according to the present invention may include both or either of the peptides of the SEQ ID Nos. 1 and 2. In this case, it is preferable that HLA class I typing is performed for a patient in advance. When the HLA class I subtype in the patient is A24, it is preferable to administer both or either of the peptides of the SEQ ID Nos. 1 and 2. On the other hand, when the patient has HLA-A02, it is preferable to administer the peptide of the SEQ ID No. 1. This cancer vaccine may contain a different kind of cancer antigenic peptide expressed in cancer cells to be treated, other than the peptides of the SEQ ID Nos. 1 and 2. In addition, the peptide may be administered in combination with, for example, an adjuvant to enhance the induction of immunity. The peptide to be administered may be modified to suppress its in vivo degradation. Alternatively, the vaccine may be administered as a DNA vaccine using, for example, an expression vector with DNA encoding the peptide rather than administering the peptide itself.

The route of administration is not specifically limited and examples include intracutaneous, subcutaneous, intravenous, and intraperitoneal administrations.

A method to obtain the peptides of the SEQ ID Nos. 1 and 2 is not specifically limited. The peptides may be isolated and purified from the cells expressing each of them. Alternatively, they may be recombinant peptides produced by recombination or peptides that are chemically synthesized using a well-known technique.

[Cancer Vaccine containing Antigen-Presenting Cells]

Antigen-presenting cells presenting the peptide of the SEQ ID No. 1 or 2 can be used as a cancer vaccine. The peptide presented on the cell surface may be unmodified or modified with sugar or phosphate. Examples of the antigen-presenting cells include dendritic cells, macrophages, B cells, and tumor cells (false antigen-presenting cells) in which a T cell stimulation factor (e.g., B7 or 4-1 BBL) and the like is forcibly expressed by, for example, gene transfer. The dendritic cells are, however, preferable in terms of their high ability to present antigens. An example of the method of isolating dendritic cells is described below but the other cells can readily be obtained using a publicly known method.

First, monocytes (hereinafter, also referred to as PBMCs) are isolated from peripheral blood of a vertebrate animal. The HLA class I typing is performed to confirm the animal carries the A24 or A02 subtype. The PBMCs used are preferably isolated from the individual to be treated, but may be isolated from an individual not to be subjected to treatment. The PBMCs are preferably CD14-positive or CD11c-positive. The method of isolating PBMCs is not specifically limited and can appropriately be determined by those skilled in the art depending on the type of PBMCs to be isolated. For example, Ficoll centrifugation can be used to isolate all PBMCs (PBMC fractions) while antibody-coated magnetic beads can be used to isolate CD14-positive PBMCs or CD11c-positive PBMCs. The isolated PBMCs can be cultured in a medium supplemented with GM-CSF and IL-4 for 5 to 7 days to induce their differentiation into dendritic cell precursors. When the differentiated dendritic cell precursors carry HLA-A24, the peptide of the SEQ ID No. 1 or 2 is added. On the other hand, when the differentiated dendritic cell precursors carry HLA-A02, the peptide of the SEQ ID No. 1 is added. The dendritic cell thus obtained is the antigen-presenting cell presenting the peptide of the SEQ ID No. 1 or 2 and is to be administered to an individual with cancer.

The route of administration is not specifically limited and examples include intracutaneous, subcutaneous, intravenous, intralymphatic, and intraperitoneal administrations. It is, however, preferable that the dendritic cells are administered directly into a cancer tissue or a lymph node in view of the fact that physiological anti-cancer immunoreactions including physiological antigen presentation on the dendritic cells occur in the cancer tissue and in the vicinity of the lymph node to which the site where the dendritic cells are administered is associated.

[Cancer Vaccine containing T Cells]

Furthermore, T cells established by the stimulation with the antigen-presenting cells presenting the peptide of the SEQ ID No. 1 or 2 can also be used as a cancer vaccine. The T cells can be obtained by co-culturing naive T cells along with the antigen-presenting cells presenting the peptide of the SEQ ID No. 1 or 2 in the presence of serum to differentiate them, into, for example, CD8-positive cytotoxic T lymphocytes (CTLs) or CD4-positive helper T cells. The T cells thus established may be administered to an individual with cancer.

The origin of the naive T cells is not specifically limited and it may be derived from, for example, peripheral blood of a vertebrate animal. The naive T cell used may be CD8-positive cells or CD4-positive cells isolated from a PBMC fraction. It is, however, preferable that the naive T cells are CD8-positive cells or CD4-positive cells mixed with other cells and components without being isolated from the PBMC fraction in terms of the efficiency of inducing CTLs. For example, when cells of a PBMC fraction are cultured in a medium supplemented with serum and the peptide consisting of the sequence 1 or 2, the PBMCs differentiate into dendritic cell precursors. The dendritic cell precursors then bind to the peptide and differentiate into dendritic cells as the antigen-presenting cells presenting this peptide. The antigen-presenting cells stimulate the CD8-positive T cells in the PBMCs to differentiate them into CTLs. Thus, the CTLs capable of recognizing the added peptide can be obtained. To this end, a culture period can appropriately be determined by those skilled in the art in a range that allows production of the CTLs. It is, however, preferable that the culture period is 4 to 10 days and more preferably, 6 days at 37° C.

The CTLs thus obtained may be isolated and used as the cancer vaccine as they are. Alternatively, they may be cultured further in the presence of interleukin such as IL-2, the antigen-presenting cell, and the peptide consisting of the sequence 1 or 2 before used as the cancer vaccine. This procedure enhances the cytotoxicity of the CTLs.

The route of their administration is not specifically limited and examples include intracutaneous, subcutaneous, intravenous, and intratumoral administrations. It is, however, preferable to use intratumoral administration because cytotoxic T lymphocytes can directly attack the cells expressing an antigen.

### EXAMPLES

### Example 1

### Expression of Snail Gene

This example shows that snail gene is hardly expressed in normal tissues and is highly expressed in cancer cell lines and cancer tissues.

==Gene Expression Analysis using RT-PCR==

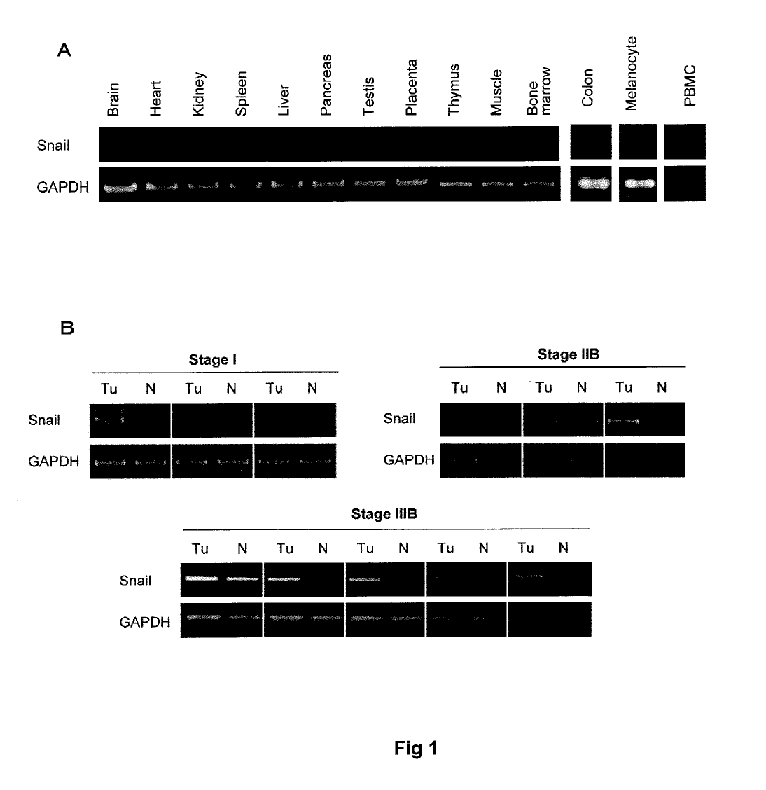

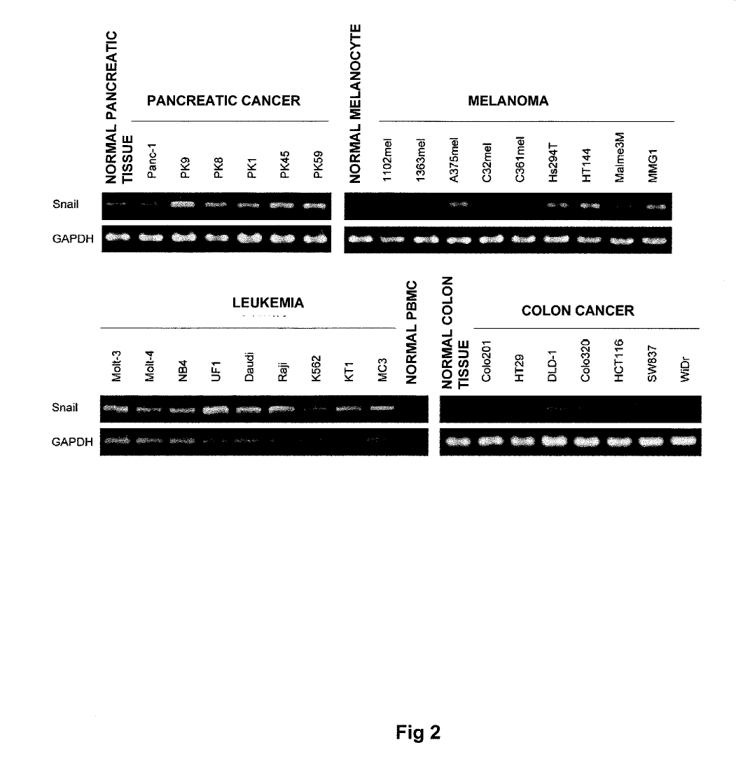

RNA was extracted from normal tissues from healthy persons, cancer tissues and a macroscopically normal part of the colon of cancer patients, and various human tumor cell lines (pancreatic cancer, melanoma, leukemia, colon cancer) (see, FIGS. 1 and 2) using RNeasy kit (Qiagen). The RNAs were reverse-transcribed with AMV into cDNAs. Next, the genes were amplified using the following primers in an iCycler (Biorad). Gene expression was then detected by electrophoresis. The expression of GAPDH gene was used as an internal standard.

| Primers for snail: | |
|---|---|
| (SEQ ID No. 5) | |
| Forward | 5′-CAGATGAGGACAGTGGGAAAGG -3′ |
| (SEQ ID No. 6) | |
| Reverse | 5′-ACTCTTGGTGCTTGTGGAGCAG -3′ |
| Primers for GAPDH: | |
| (SEQ ID No. 7) | |
| Forward | 5′- GTCAACGGATTTGGTCGTATT -3′ |
| (SEQ ID No. 8) | |
| Reverse | 5′- ATCACTGCCACCCAGAAGACT -3′ |

As shown in FIG. 1A, weak expression of the snail gene was detected in the normal tissues from the placenta, testis, melanocyte, and colon of the healthy person while no expression was detected in those of the brain, heart, kidney, spleen, liver, pancreas, thymus, muscle, bone marrow, and peripheral blood monocyte (PBMC).

In addition, FIG. 1B shows the expression of the snail gene in colon cancer tissues (Tu) obtained from the patients with colon cancer at different stages and the normal tissue (N) (macroscopically determined as normal) of the colon of the same cancer patients. A higher expression of the snail gene was detected in the tissues of the colon cancer at all the stages I, IIB, and IIIB determined according to the American Joint Committee on Cancer (AJCC) staging system commonly used worldwide, as compared to the expression in the normal tissue.

Furthermore, as shown in FIG. 2, the snail gene was less expressed in the cell lines from the pancreatic cancer, melanoma, leukemia, and colon cancer than in the normal pancreatic tissue, melanocyte, peripheral blood monocyte, and colon tissue.

Thus, the snail gene is expressed specifically in the cancer tissues. Therefore, the expression of the snail gene can be used for the diagnosis of cancers, and that therapies targeted to the Snail as a cancer antigen can be applied to patients with various kinds of cancers because of its less adverse effects on the organs.

### Example 2

### Induction of CTLs using HLA-A24-Positive PBMCs from Healthy Persons

This example shows that CTLs that recognize and activate Snail 1 and Snail 3 peptides can be induced from HLA-A24-positive PBMCs isolated from healthy persons by using these peptides.

First, PBMCs were isolated from healthy, HLA-A24-positive persons (A, B) as follows. To the peripheral blood obtained, 4% sodium citrate was added in an amount equal to one tenth of the amount of the blood. The mixture was overlaid on Ficoll-Paque (Amersham) and centrifuged (1500 rpm, 20 minutes, room temperature). The intermediate layer containing PBMCs was separated as a PBMC fraction. The PBMCs of 2.5×10⁷ were suspended in an RPMI1640 medium (Invitrogen) supplemented with 20 ml of 10% fetal bovine serum (FCS), to which 10 μg/ml each of the peptides (Snail 1 to 3) was added. The mixture was incubated with stimulation for 6 days at 37° C. in the presence of 5% CO₂. This incubation with stimulation leads to the differentiation of the CTLs responsive to each antigenic peptide. These CTLs were separated using antibody-coated MACS magnetic beads available from Myltenyi.

An HERV-H env peptide (SEQ ID No. 10) and a NY-ESO-1 peptide (SEQ ID No. 11) were used as positive controls. It is known that the HERV-H env peptide and the NY-ESO-1 peptide are presented by HLA-A24-positive antigen-presenting cells and induce CTLs from CD8-positive T cells, and that the CTLs obtained are capable of recognizing the HERV-H env peptide and the NY-ESO-1peptide to produce gamma interferon.

| | | |
|---|---|---|
| Snail 1: | KMHIRSHTL | (SEQ ID No. 1) |
| Snail 2: | KAFSRPWLL | (SEQ ID No. 9) |
| Snail 3: | RTFSRMSLL | (SEQ ID No. 2) |
| HERV-H env: | SYLHHTINL | (SEQ ID No. 10) |
| NY-ESO-1: | LLMWITQCF | (SEQ ID No. 11) |

The 1×10⁶ CTLs obtained by the incubation with stimulation were co-cultured with the antigen-presenting cells in an RPMI1640 medium supplemented with 10% FCS in the presence of IL-2 (100 U/ml, Peprotech) and the same peptide (0.1, 1 or 10 μg/ml) as the one used for the differentiation of the CTLs to stimulate the CTLs with the peptide. The antigen-presenting cells used were prepared by incubating in suspension the PBMCs (total cells) obtained from the same healthy persons from whom the PBMCs used for the differentiation of the CTLs had been obtained, in an RPMI1640 medium supplemented with 10 ml of 10% FCS and 10 μg/ml of mitomycin C for 2 hours (37° C., 5% CO) to inactivate the cells, and then washing them with the RPMI1640 medium. When the antigen-presenting cells presenting the peptide stimulate the CTLs with the HLA-A24-restricted peptide, the CTLs produce gamma interferon. The gamma interferon contained in the supernatant was measured using a human Cytometric Bead Array kit (BD Biosciences) to determine whether the CTLs can recognize the peptide presented by the antigen-presenting cells.

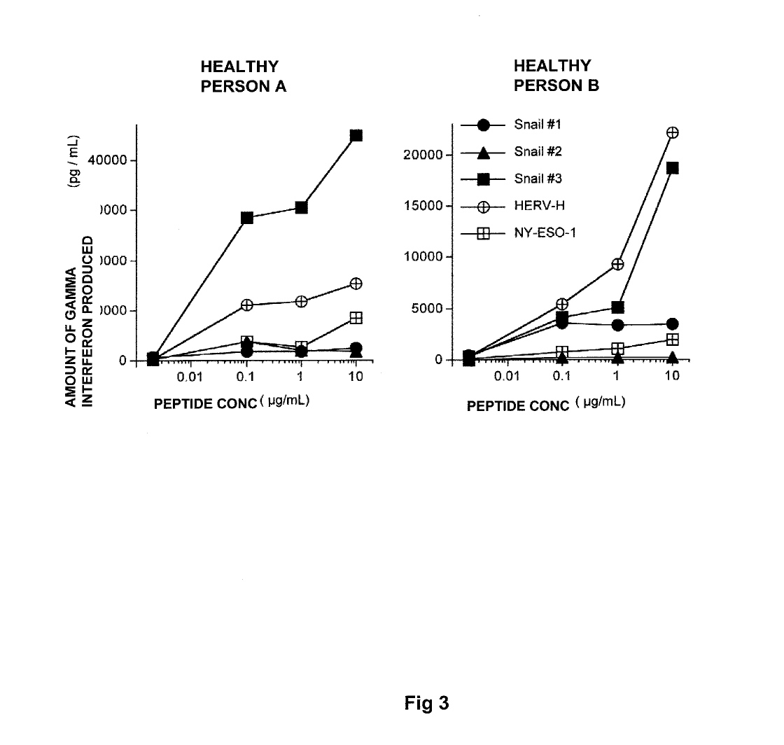

FIG. 3 shows the amount of the gamma interferon produced by the CTLs in healthy persons A and B when the CTLs were co-cultured with the antigen-presenting cells with the addition of the peptide at a concentration of 0.1 to 10 μg/ml. The leftmost point in the graph for the healthy person A represents a peptide-free group without any peptide added, while the leftmost point in the graph for the healthy person B represents a negative control group with the addition of 10 μg/ml of a negative control peptide (KSPWFTTL, mouse retrovirus antigen p15e peptide, SEQ ID No. 12). In the peptide-free and negative control groups, only about 100 to 500 pg/ml of gamma interferon was produced. On the contrary, in the groups stimulated with the Snail 1 peptide, the Snail 3 peptide or the positive control (HERV-H env peptide or NY-ESO-1 peptide), the amount of the gamma interferon produced was significantly increased along with the increase of the peptide concentration (p<0.01, t-study).

As shown in FIG. 3, the amount of the gamma interferon produced reached the maximum when the peptide concentration was 10 μg/ml. The amount of the gamma interferon produced in the healthy person A or B was significantly larger in the groups with the addition of 10 μg/ml of the Snail 1 peptide or the Snail 3 peptide (p<0.01, t-study) as compared to the peptide-free or negative control group, and was comparable or larger as compared to the groups of the positive controls, the HERV-H env peptide and the NY-ESO-1 peptide.

Thus, the HLA-A24-positive PMBCs from the healthy persons can be used to obtain the antigen-presenting cells presenting the Snail 1 peptide (SEQ ID No. 1) or the Snail 3 peptide (SEQ ID No. 2) on their cell surface. In addition, stimulation of the CD8-positive T cells with this antigen-presenting cell leads to the differentiation of the CTLs capable of recognizing the peptides presented by the HLA-A24-positive antigen-presenting cells.

### Example 3

### Tumor Cytotoxicity of CTLs recognizing Snail 3

This example shows that CTLs obtained by culturing PBMCs with stimulation with a Snail 3 peptide have cytotoxicity against snail-positive tumor cells in an HLA-dependent manner.

CTLs prepared by culturing PBMCs obtained from blood of a healthy, HLA-A24-positive person with stimulation with the Snail 3 peptide (see Example 2, CTLs recognizing Snail 3) were mixed with a human colon cancer cell line COLO320 (HLA-A24-positive, snail-positive, and NY-ESO-1-positive) as a target cell in a ratio of CTL:COLO320=6.25:1, 12.5:1, 25:1 or 50:1. The mixture was then incubated for 6 hours at 37° C. under the condition of 5% CO₂ in RPMI1640 medium supplemented with 10% FCS. The killed cells were detected using Immunocyto Cytotoxity Detection Kit (MBL) and the tumor-specific cytotoxicity was calculated according to the protocol appended to the kit. In this example, the PMBCs were cultured with stimulation with the NY-ESO-1 peptide instead of the Snail 3 peptide as the positive control.

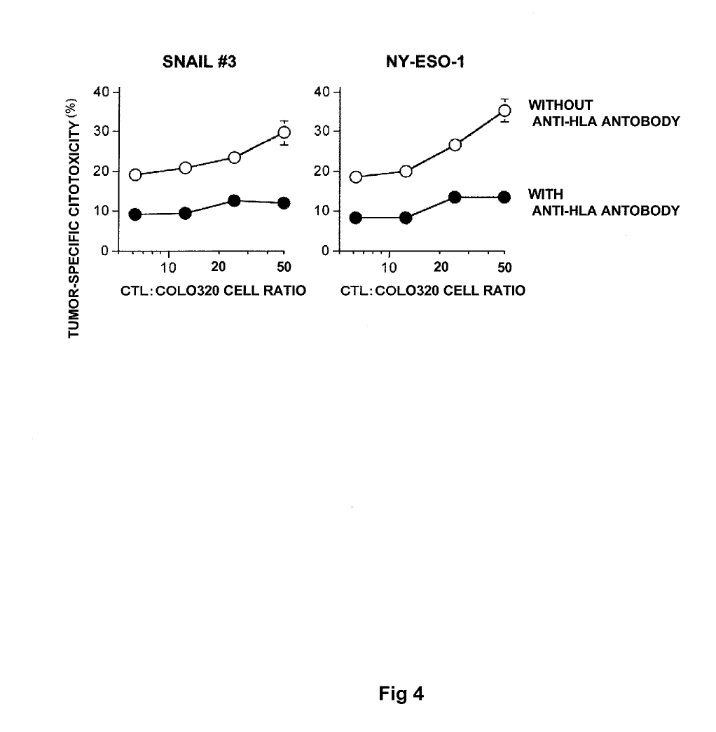

As shown in FIG. 4, the tumor-specific cytotoxicity of the CTLs recognizing Snail 3 was comparable to that of the positive control, i.e., the CTLs recognizing NY-ESO-1. The tumor-specific cytotoxicity was increased with the increase in the amount of the CTLs mixed. Thus, the CTLs obtained by culturing the PMBCs with stimulation with the Snail 3 peptide have the cytotoxicity against the tumor cells expressing snail (see, graphs of the points designated by open circles in FIGS. 4A and 4B).

In addition, in order to show that the recognition of the Snail antigen on the tumor cells by the CTLs recognizing Snail 3 is HLA-dependent, the CTLs recognizing Snail 3 were co-cultured with the COLO320 cells in the presence of an anti-HLA antibody (HLA neutralizing antibody, BioLegend, final concentration of 10 μg/ml).

As shown in FIG. 4, the tumor-specific cytotoxicity of the CTLs recognizing Snail 3 was reduced significantly in the group with the addition of the anti-HLA antibody (see, graphs of the points designated by solid circles in FIGS. 4A and 4B), as compared to the group without any anti-HLA antibody (see, the graphs of the points designated by open circles in FIGS. 4A and 4B). This result indicates that the CTLs recognizing Snail 3 recognize the Snail antigen on the tumor cells in an HLA-dependent manner when the CTLs recognizing Snail 3 specifically kill tumor cells.

These results indicate that the stimulation of the HLA-A24-positive PMBCs obtained from a healthy person with the Snail 3 peptide leads to the induction of the CTLs having cytotoxicity against a snail antigen-positive target cell, and that the CTLs recognize the Snail antigen on the target cells in an HLA-dependent manner.

### Example 4

### Differentiation of CTLs using HLA-A02-Positive PBMCs from Healthy Persons

This example shows that CTLs whose differentiation has been induced using the Snail 1 peptide can recognize the cells presenting the Snail 1 peptide in an HLA-A02-dependent manner.

First, according to the method described in Example 2, PBMCs were isolated from blood obtained from healthy, HLA-A02-positive persons (D, E). The PBMCs were cultured with the Snail 1 peptide. In this example, the HERV-H env peptide was used as the positive control and the NY-ESO-1 peptide was used as the negative control. It is known that the HERV-H env peptide is presented by HLA-A02-positive antigen-presenting cells and induces CTLs from CD8-positive T cells, and that the CTLs obtained are capable of recognizing the HERV-H env peptide to produce gamma interferon. It is also known that the NY-ESO-1 peptide does not bind HLA-A02-positive antigen-presenting cells and CTL is not induced with this peptide. The CTLs thus obtained were stimulated for 24 hours in an RPMI1640 medium supplemented with 10% FCS and each peptide (10 μg/ml) in the presence of IL-2 and the HLA-A02-positive antigen-presenting cell prepared as in the case of the Example 2. When the antigen-presenting cells presenting the peptide stimulate the CTLs with the HLA-A02-restricted peptide, the CTLs produce gamma interferon. The gamma interferon contained in the supernatant was measured using a human Cytometric Bead Array kit (BD Biosciences) to determine whether the CTLs can recognize the antigen-presenting cell presenting the peptide.

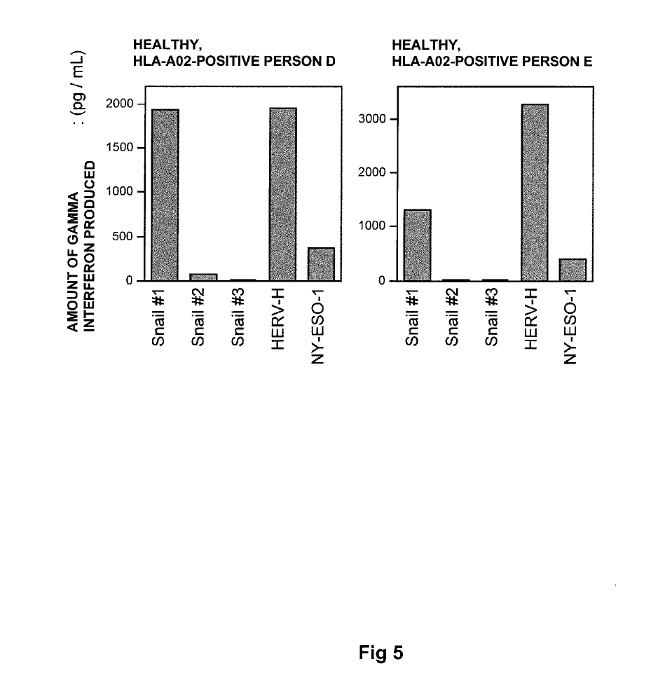

FIG. 5 shows the amount of gamma interferon produced by the CTLs prepared from the PBMCs obtained from the healthy persons D and E. In the healthy persons D and E, the amount of the gamma interferon produced was significantly larger in the groups stimulated with the Snail 1 peptide and the HERV-H env peptide (positive control) (p<0.01, t-study) as compared to the group stimulated with the negative control, i.e., the NY-ESO-1 peptide. On the other hand, the amount of the gamma interferon produced was smaller in the groups stimulated with the Snail 2 peptide or the Snail 3 peptide as compared to the negative control.

Thus, the Snail 1 peptide (SEQ ID No. 1) is presented on the antigen-presenting cells derived from the HLA-A02-positive PBMCs obtained from a healthy person and induces the CTLs that recognize the peptide from the CD8-positive T cells. Furthermore, the CTLs can recognize the Snail 1 peptide on the HLA-A02-positive presenting cells.

### INDUSTRIAL APPLICABILITY

The present invention provides the peptides that can efficiently induce cancer immunity, the antigen-presenting cells presenting one of these peptides on their cell surface, the T cells that are induced by the antigen-presenting cells, and the cancer vaccines comprising these peptides, expression vectors capable of expressing the respective peptides, antigen-presenting cells presenting these peptides, or T cells induced by the antigen-presenting cells as well as a method of treating and preventing cancers using the cancer vaccine(s).

1. A peptide consisting of a sequence of SEQ ID No. 1 (KMHIRSHTL) or SEQ ID No. 2 (RTFSRMSLL).

2. An antigen-presenting cell presenting a peptide consisting of a sequence of SEQ ID No. 1 or 2 on its cell surface.

3. A T cell induced by the antigen-presenting cell according to claim 2, the T cell being capable of recognizing a cancer cell expressing a Snail antigen.

4. The T cell according to claim 3, wherein the T cell is cytotoxic T lymphocyte.

5. The T cell according to claim 3, wherein the cancer cell is a pancreatic cancer cell, a melanoma cell, a leukemia cell or a colon cancer cell.

6. A cancer vaccine containing one or more peptides selected from SEQ ID Nos. 1 and 2, an expression vector capable of expressing either of the peptides, and the antigen-presenting cell according to claim 2.

7. The cancer vaccine according to claim 6, wherein the cancer vaccine is against a cancer cell expressing a Snail protein.

8. The cancer vaccine according to claim 6, wherein the cancer is pancreatic cancer, melanoma, leukemia or colon cancer.

9. A method of treating or preventing a cancer of a vertebrate animal using the cancer vaccine according to claim 8.

10. A cancer vaccine containing one or more peptides selected from SEQ ID Nos. 1 and 2, an expression vector capable of expressing either of the peptides, and the T cell according to claim 3.

11. The cancer vaccine according to claim 10, wherein the cancer vaccine is against a cancer cell expressing a Snail protein.

12. The cancer vaccine according to claim 10, wherein the cancer is pancreatic cancer, melanoma, leukemia or colon cancer.

13. A method of treating or preventing a cancer of a vertebrate animal using the cancer vaccine according to claim 12.


SEQUENCE LISTING (12 sequences)

SEQ ID NO: 1 - protein, 9 aa, Homo sapiens
KMHIRSHTL

SEQ ID NO: 2 - protein, 9 aa, Homo sapiens
RTFSRMSLL

SEQ ID NO: 3 - DNA, 1708 bp, Homo sapiens
ggcacggcctagcgagtggttcttctgcgctactgctgcgcgaatcggcgaccccagtgcctcgaccactatgccgcgctctttcctcgtcaggaagccctccgaccccaatcggaagcctaactacagcgagctgcaggactctaatccagagtttaccttccagcagccctacgaccaggcccacctgctggcagccatcccacctccggagatcctcaaccccaccgcctcgctgccaatgctcatctgggactctgtcctggcgccccaagcccagccaattgcctgggcctcccttcggctccaggagagtcccagggtggcagagctgacctccctgtcagatgaggacagtgggaaaggctcccagccccccagcccaccctcaccggctccttcgtccttctcctctacttcagtctcttccttggaggccgaggcctatgctgccttcccaggcttgggccaagtgcccaagcagctggcccagctctctgaggccaaggatctccaggctcgaaaggccttcaactgcaaatactgcaacaaggaatacctcagcctgggtgccctcaagatgcacatccgaagccacacgctgccctgcgtctgcggaacctgcgggaaggccttctctaggccctggctgctacaaggccatgtccggacccacactggcgagaagcccttctcctgtccccactgcagccgtgccttcgctgaccgctccaacctgcgggcccacctccagacccactcagatgtcaagaagtaccagtgccaggcgtgtgctcggaccttctcccgaatgtccctgctccacaagcaccaagagtccggctgctcaggatgtccccgctgaccctcgaggctccctcttcctctccatacctgcccctgcctgacagccttccccagctccagcaggaaggaccccacatccttctcactgccatggaattccctcctgagtgccccacttctggccacatcagccccacaggactttgatgaagaccattttctggttctgtgtcctctgcctgggctctggaagaggccttcccatggccatttctgtggagggagggcagctggcccccagccctgggggattcctgagctggcctgtctgcgtgggtttttgtatccagagctgtttggatacagctgctttgagctacaggacaaaggctgacagactcactgggaagctcccaccccactcaggggaccccactcccctcacacacacccccccacaaggaaccctcaggccaccctccacgaggtgtgactaactatgcaataatccacccccaggtgcagccccagggcctgcggaggcggtggcagactagagtctgagatgccccgagcccaggcagctatttcagcctcctgtttggtggggtggcacctgtttcccgggcaatttaacaatgtctgaaaagggactgtgagtaatggctgtcacttgtcgggggcccaagtggggtgctctggtctgaccgatgtgtctcccagaactattctgggggcccgacaggtgggcctgggaggaagatgtttacatttttaaaggtacactggtatttatatttcaaacattttgtatcaaggaaacgttttgtatagttatatgtacagtttattgatattcaataaagcagttaatttatatattaaaaaaaaaaaaaaaaaa

SEQ ID NO: 4 - protein, 264 aa, Homo sapiens
MPRSFLVRKPSDPNRKPNYSELQDSNPEFTFQQPYDQAHLLAAIPPPEILNPTASLPMLIWDSVLAPQAQPIAWASLRLQESPRVAELTSLSDEDSGKGSQPPSPPSPAPSSFSSTSVSSLEAEAYAAFPGLGQVPKQLAQLSEAKDLQARKAFNCKYCNKEYLSLGALKMHIRSHTLPCVCGTCGKAFSRPWLLQGHVRTHTGEKPFSCPHCSRAFADRSNLRAHLQTHSDVKKYQCQACARTFSRMSLLHKHQESGCSGCPR

SEQ ID NO: 5 - DNA, 22 bp, Artificial Sequence
  Primer
cagatgaggacagtgggaaagg

SEQ ID NO: 6 - DNA, 22 bp, Artificial Sequence
  Primer
actcttggtgcttgtggagcag

SEQ ID NO: 7 - DNA, 21 bp, Artificial Sequence
  Primer
gtcaacggatttggtcgtatt

SEQ ID NO: 8 - DNA, 21 bp, Artificial Sequence
  Primer
atcactgccacccagaagact

SEQ ID NO: 9 - protein, 9 aa, Homo sapiens
KAFSRPWLL

SEQ ID NO: 10 - protein, 9 aa, Homo sapiens
SYLHHTINL

SEQ ID NO: 11 - protein, 9 aa, Homo sapiens
LLMWITQCF

SEQ ID NO: 12 - protein, 8 aa, Mus musculus
KSPWFTTL